(19) United States
(12) Patent Application Publication
(10) Pub. No.: US 2012/0277588 A1
(43) Pub. Date: Nov. 1, 2012

(54) SYSTEMS AND METHODS FOR FUSING SENSOR AND IMAGE DATA FOR THREE-DIMENSIONAL VOLUME RECONSTRUCTION

(75) Inventors: DIRK RYAN PADFIELD, ALBANY, NY (US); KEDAR PATWARDHAN, LATHAM, NY (US); KIRK WALLACE, GREEN ISLAND, NY (US)

(73) Assignee: GENERAL ELECTRIC COMPANY, SCHENECTADY, NY (US)

(21) Appl. No.: 13/094,628

(22) Filed: Apr. 26, 2011

(51) Int. Cl. A61B 8/14

(52) U.S. Cl. 600/443

(57) ABSTRACT

An imaging system for generating three-dimensional (3D) images includes an imaging probe for acquiring two-dimensional (2D) image data of a region of interest. A sensor is coupled with the imaging probe to determine positional data related to a position of the imaging probe. A position determination module utilizes the image data acquired with the imaging probe and the positional data determined by the sensor to calculate a probe location with respect to the acquired 2D image data. An imaging module is configured to reconstruct a 3D image of the region of interest based on the 2D image data and the determined probe locations.

### BACKGROUND

The subject matter disclosed herein relates to imaging systems, and more particularly, to systems and methods for generating three-dimensional (3D) images.

Two-dimensional (2D) imaging systems may be utilized to generate 3D images. In some systems, an imaging probe, such as an ultrasound probe, is equipped with a sensor to track the location of the probe as the probe is moved about a subject to acquire 2D images of a region of interest. The sensor may include a position tracking device, similar to a Global Positioning System (GPS) tracking device, and/or an accelerometer to track both the position and the orientation of the probe. The positional data acquired by the sensor is utilized to reconstruct 3D images from the 2D images acquired with the probe. However, the sensor may be subject to errors over time. In particular, as the imaging probe is moved about the subject, errors may accumulate with respect to the positional data. Accordingly, over time, the positional data becomes less accurate. As a result, an operator may be required to frequently re-calibrate the sensor by holding the sensor still for a period of time. This delay reduces the efficiency and throughput for scans being performed by the probe.

Additionally, in the absence of a position sensor, when reconstructing 3D images with the 2D images acquired by the imaging probe, an imaging module may align or overlap a series of 2D images acquired with the imaging probe to reconstruct the 3D image. However, such 3D image reconstruction is subject to errors because the imaging module lacks a framework within which to reconstruct the 3D image. Specifically, determination of the alignment of the images can become difficult because the alignment requires closely spaced images with overlap. When the probe moves in elevation or rotates, there is almost no alignment and the alignment of the images becomes even more difficult. The lack of a framework may lead to blurred and/or jagged images in the 3D reconstruction.

### SUMMARY

In one embodiment, an imaging system for generating three-dimensional (3D) images is provided. The system includes an imaging probe for acquiring two-dimensional (2D) image data of a region of interest. A sensor is coupled with the imaging probe to determine positional data related to a position of the imaging probe. A position determination module utilizes the image data acquired with the imaging probe and the positional data determined by the sensor to calculate a probe location with respect to the acquired 2D image data. An imaging module is configured to reconstruct a 3D image of the region of interest based on the 2D image data and the determined probe locations.

In another embodiment, a method for generating three-dimensional (3D) images is provided. The method includes acquiring two-dimensional (2D) image data of a region of interest with an imaging probe. Positional data related to a position of the imaging probe is determined with a sensor coupled with the imaging probe. A probe location with respect to the acquired 2D image data is calculated with the imaging data acquired with the imaging probe and the positional data determined by the sensor. A 3D image of the region of interest is reconstructed based on the 2D image data and the determined probe locations.

In another embodiment, a non-transitory computer readable storage medium for generating three-dimensional (3D) images using a processor is provided. The non-transitory computer readable storage medium includes instructions to command the processor to acquire two-dimensional (2D) image data of a region of interest with an imaging probe. Positional data related to a position of the imaging probe is determined with a sensor coupled with the imaging probe. A probe location with respect to the acquired 2D image data is calculated with the imaging data acquired with the imaging probe and the positional data determined by the sensor. A 3D image of the region of interest is reconstructed based on the 2D image data and the determined probe locations.

### BRIEF DESCRIPTION OF THE DRAWINGS

The presently disclosed subject matter will be better understood from reading the following description of non-limiting embodiments, with reference to the attached drawings, wherein below:

FIG. 1 is a schematic block diagram of an imaging system formed in accordance with an embodiment.

FIG. 2 is a schematic block diagram of the imaging system shown in FIG. 1 including a transmitter/receiver.

FIG. 3 is a diagram illustrating an imaging probe and sensor in connection with which various embodiments may be implemented.

FIG. 4 is a flowchart of a method of reconstructing a 3D image in accordance with an embodiment.

FIG. 5 is a graph of the root mean square data corresponding to acquired image slices used in accordance with an embodiment.

FIG. 6 is an exemplary representation of error over time in 3D image reconstruction in accordance with an embodiment.

FIG. 7 illustrates a hand carried or pocket-sized ultrasound imaging system formed in accordance with an embodiment.

FIG. 8 illustrates an ultrasound imaging system formed in accordance with an embodiment and provided on a moveable base.

FIG. 9 illustrates a 3D-capable miniaturized ultrasound system formed in accordance with an embodiment.

### DETAILED DESCRIPTION

The foregoing summary, as well as the following detailed description of certain embodiments, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors, controllers, circuits or memories) may be implemented in a single piece of hardware or multiple pieces of hardware. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Various embodiments provide an imaging system that utilizes image information to compensate for positional data related to a position of an imaging probe and used during image reconstruction of a three-dimensional (3D) image from two-dimensional (2D) image data. In particular, the positional data may be utilized to align a reconstructed 3D image, such as of a region of interest. The imaging data is used to correct, adjust, or align the 3D image. In general, the positional data is subject to greater errors over time because the measurements may drift especially when the sensor is acquires differential measurements, while errors in the imaging data decrease over time because alignment of images is more accurate when more of the 3D volume has already been reconstructed. As such, the positional data and the imaging data can be weighted to reduce errors in the reconstructed 3D image.

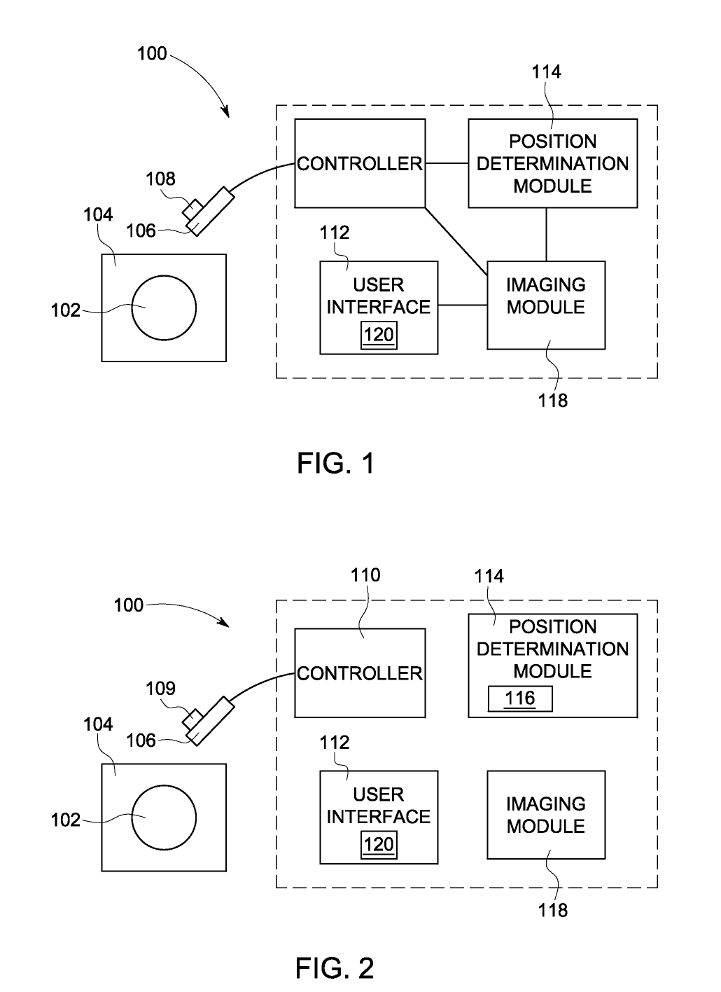

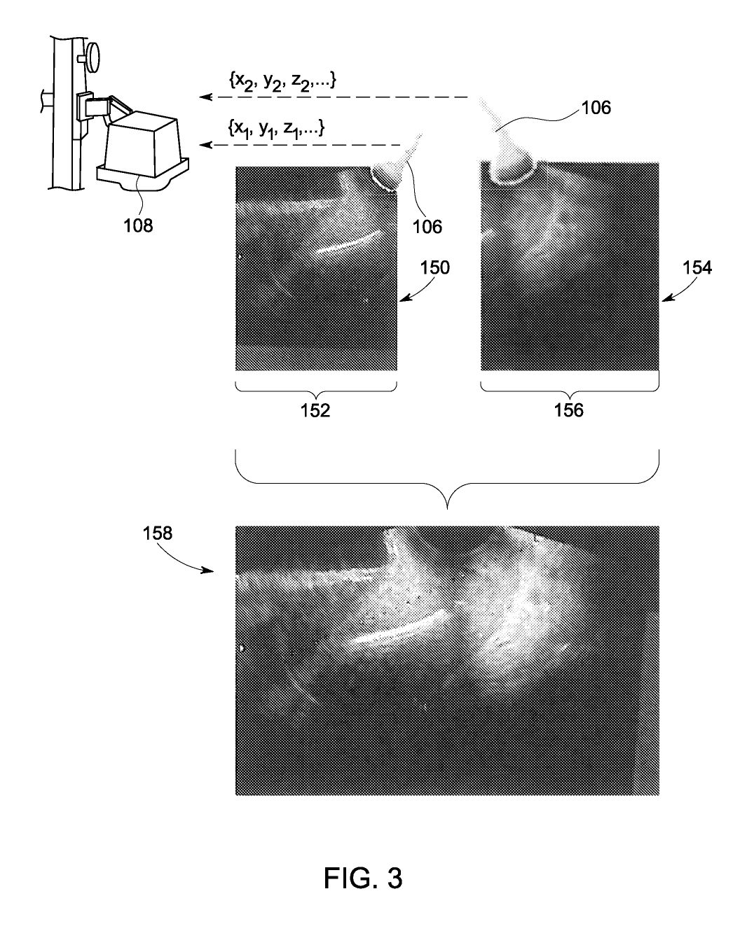

FIG. 1 is a schematic block diagram of an imaging system 100 formed in accordance with an embodiment. FIG. 2 is a schematic block diagram of the imaging system 100 including a transmitter/receiver 116, as discussed below. The imaging system 100 is configured to generate a 3D image of a region of interest 102, for example an anatomy of interest, of a subject 104 (e.g. a patient). The imaging system 100 generates the 3D image by reconstructing 2D imaging data. It should be noted that as used herein, imaging data and image data both generally refer to data used to reconstruct an image.

In an exemplary embodiment, the 2D imaging data is acquired with an imaging probe 106. In one embodiment, the imaging probe 106 may be a hand-held ultrasound imaging probe. Alternatively, the imaging probe 106 may be an infrared-optical tomography probe. The imaging probe 106 may be any suitable probe for acquiring 2D images in another embodiment. The imaging system 100 reconstructs the 3D image based on 2D imaging data. The imaging probe 106 is illustrated as being mechanically coupled to the imaging system 100. Alternatively, the imaging probe 106 may be in wireless communication with the imaging system 100.

The imaging probe 106 includes a sensor 108 coupled therewith. For example, the sensor 108 may be a differential sensor. In one embodiment, the sensor 108 is externally coupled to the imaging probe 106. The sensor 108 may be formed integrally with and positioned in a housing of the imaging probe 106 in other embodiments. In one embodiment, the sensor 108 may be an accelerometer, for example, a three-axis accelerometer, a gyroscope, for example, a three-axis gyroscope, or the like that determines the x, y, and z coordinates of the imaging probe 106. In another embodiment, the sensor 108 may be a tracking device, similar to a Global Positioning System (GPS) tracking device or the like. The tracking device receives and transmits signals indicative of a position thereof. The sensor 108 is used to acquire positional data of the imaging probe 106. For example, the sensor 108 determines a position and an orientation of the imaging probe 106. Other position sensing devices may be used, for example, optical, ultrasonic, or electro-magnetic position detection systems.

A controller 110 is provided to control scan parameters of the imaging probe 106. For example, the controller 110 may control acquisition parameters (e.g. mode of operation) of the imaging probe 106. In another embodiment, the controller 110 may control other scan parameters (e.g. gain, frequency, etc.) of the imaging probe 106. The controller 110 may control the imaging probe 106 based on scan parameters provided by an operator at a user interface 112. The operator may set the scan parameters of the imaging probe prior to image acquisition with the imaging probe 106. In one embodiment, the operator may adjust the scan parameters of the imaging probe during image acquisition.

The imaging system 100 includes a position determination module 114. The position determination module 114 determines a position and/or orientation of the imaging probe 106 based on data received from the sensor 108, as well as image data as discussed in more detail herein. In the embodiment illustrated in FIG. 1, the position determination module 114 receives positional data determined by the sensor 108. In the embodiment illustrated in FIG. 2, the position determination module 114 includes the transmitter/receiver 116 to direct signals to a sensor, which in this embodiment is a tracking device 109. The tracking device 109 transmits signals back to the transmitter/receiver 116 to indicate a position and orientation of the imaging probe 106.

The position determination module 114 may include a processor or computer that utilizes the positional data and image data to determine probe locations, which are used as part of the 3D image reconstruction process for reconstructing the imaging data acquired by the imaging probe. In particular, the 2D imaging data is aligned based on the positional data and the image data. In one embodiment, the 2D imaging data may be aligned based on positional data from the sensor 108 and of landmarks in the 2D imaging data. The position determination module 114 utilizes the data to align reconstructed 3D images of the region of interest 102.

An imaging module 118 is provided to reconstruct the 3D image based on the 2D imaging data. The imaging module 118 may include a processor or computer that reconstructs the 3D image. The 2D imaging data may include overlapping and adjacent 2D image slices. The imaging module 118 combines (e.g. aligns, shifts, reorients, etc.) the 2D image slices to reconstruct the 3D image. In an exemplary embodiment, the imaging module 118 reconstructs the 3D image, which may be within a 3D image boundary generated as described herein. In one embodiment, the imaging data is used to compensate for errors in the positional data from the sensor 108 by correcting, aligning, or adjusting the 2D image planes to reduce the errors from the positional data, which can increase over time. The information from the image data also may be used by the imaging module 118 to provide an increased level of granularity in the reconstructed 3D image.

In general, positional data determined by the sensor 108 is subject to an increasing amount of error over time. Conversely, the overall error associated with the aggregated imaging data acquired by the imaging probe 106 decreases over time. Accordingly, the imaging system 100 utilizes both the positional data and the imaging data for reconstruction of the 3D image. In one embodiment, the positional data and the imaging data used to compensate for errors in the positional data are weighted throughout the image acquisition time based on the data experiencing the least amount of error or determined to be more reliable. For example, the positional data and the image data may be weighted using fusion methods, such a Kalman Filtering. A weighting ratio of the use of imaging data to positional data for position determination generally increases over time as the sensor 108 becomes subject to more error and the imaging data becomes subject to less error. Accordingly, by utilizing a combination of positional data and imaging data, positional or alignment errors in the reconstructed 3D image is reduced or minimized, as described in more detail with respect to FIG. 6.

In one embodiment, a display 120 is provided at the user interface 112. The reconstructed 3D image may be displayed on the display 120 during the image acquisition. Alternatively, the reconstructed 3D image may be displayed as a final image after the completion of image acquisition. It should be noted, that the user interface 112 is illustrated as being embodied in the imaging system 100. The user interface 112 may be part of a separate workstation (not shown) that is provided remotely from the imaging system 100 in alternative embodiments.

FIG. 3 is a diagram illustrating an imaging probe 106 and sensor 108 in which various embodiments may be implemented. The sensor 108 is positioned remote from the imaging probe 106. The sensor 108 transmits signals to and receives signals from the tracking device 109 (shown in FIG. 2) coupled with the imaging probe 106 to determine a position of the imaging probe 106. Optionally, the sensor 108 may include the transmitter/receiver 116 that communicates with the tracking device 109 (shown in FIG. 2) coupled with the imaging probe 106 or the sensor 108 may be coupled with the imaging probe 106 to communicate with the transmitter/receiver 116 located remote from the imaging probe 106. In some embodiments, no tracking device is provided and the sensor 108 determines the location, position, or orientation of the imaging probe 106.

FIG. 3 illustrates the imaging probe 106 in a first position 150 to acquire first image data 152 and in a second position 154 to acquire second image data 156. In the first position 150, the imaging probe 106 has the coordinates x₁, y₁, and z₁. In the second position 154, the imaging probe 106 has the coordinates x₂, y₂, and z₂. The positions 150 and 154 generally represent two locations/orientations of the imaging probe 106 during a free-hand scan. The coordinates of the first position 150 and the second position 154 of the imaging probe 106 (e.g. relative spatial positions or orientations) may be utilized to align the first image data 152 and the second image data 156 in combination with the image data to form a 3D image 158. It should be noted that the imaging probe 106 may also have angular coordinates, for example, yaw, pitch and roll; azimuth, elevation, and roll; or phi, theta, and psi. The imaging probe 106 may also have a velocity, acceleration, direction, or the like. Accordingly, this positional information, among other positional information, may be measured by the sensor 108.

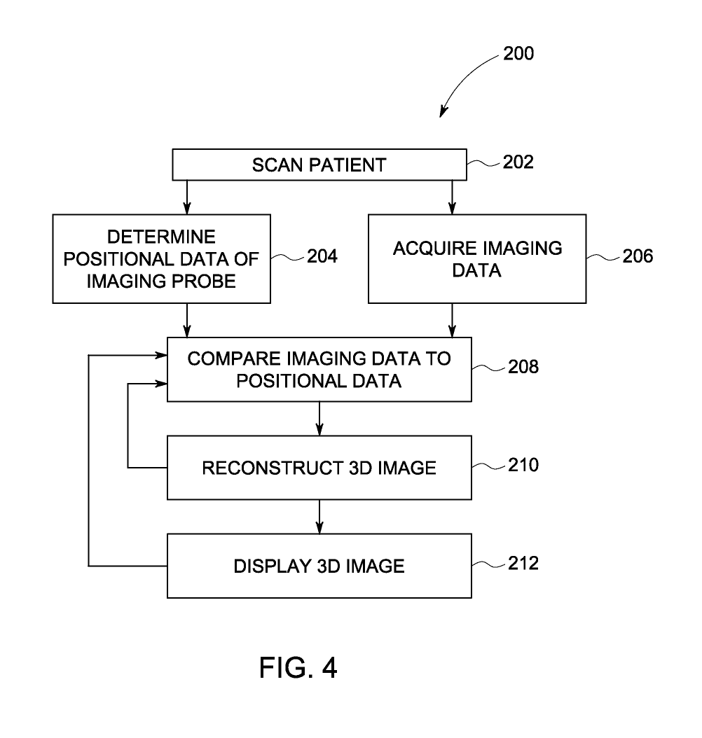

FIG. 4 is a flowchart of a method 200 of reconstructing a 3D image in accordance with an embodiment. It should be noted that the method 200 may be performed by processors and/or computers of the imaging system 100 (shown in FIGS. 1 and 2). Additionally, the method 200 may be performed by a tangible non-transitory computer readable medium. The method 200 includes scanning a patient at 202. In an exemplary embodiment, the patient is scanned free-hand with the imaging probe 106 (shown in FIGS. 1 and 2), such as a 2D ultrasound imaging probe. Initially, the patient may be scanned with broad strokes or sweeps to image a boundary of a region of interest, which is later updated with image data from additional localized scanning operation.

At 204, a position of the imaging probe 106 is determined by the position determination module 114 (shown in FIGS. 1 and 2). The position determination module 114 receives positional data from the sensor 108 (shown in FIGS. 1 and 2) or tracking device 109 to determine a position and orientation of the imaging probe 106 during the scan, for example, the initial scan. The position determination module 114 provides positional data to the imaging module 118 that is used to align a reconstructed image as described below. The position determination module 114 may optionally display the 3D reconstructed image as the positional data is determined. In particular, the position determination module 114 may display boundaries of the 3D image. An operator may update scan parameters based on the displayed boundaries.

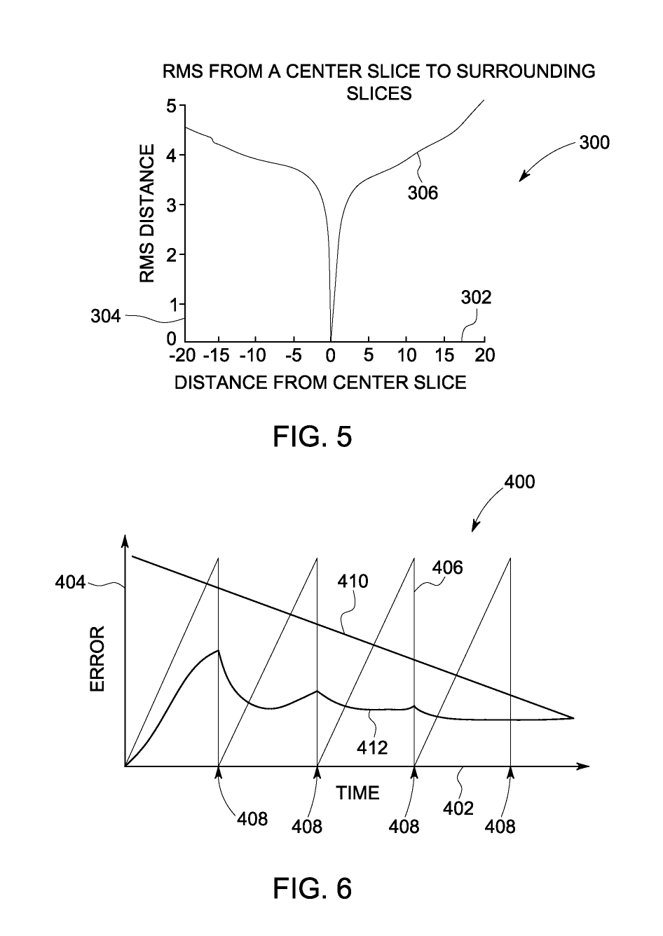

The imaging module 118 (shown in FIGS. 1 and 2) also acquires imaging data from the imaging probe 106 at 204. The imaging data is acquired simultaneously or concurrently with the positional data. The imaging module 118 reconstructs the 3D image based on the imaging data. The imaging module 118 utilizes the positional data from the sensor 108, as well as image data, for example from a plurality of imaging metrics to align the 2D image slices forming the 3D image during the image reconstruction process. For example, in addition to the positional information from the sensor 108, the imaging module 118 may align and reconstruct the 3D image based on a root mean square of a distance between 2D image slices, as illustrated in FIG. 5. Optionally, the imaging module 118 may utilize correlations or mutual information from the 2D image slices to also align the reconstruct the 3D image. In one embodiment, the alignment of the 3D reconstruction is performed utilizing landmarks within the 2D image slices, histograms of the 2D image slices, and/or speckle correlation between the 2D image slices, in addition to using the positional data from the sensor 108.

At 208, the imaging system 100 compares the positional data and the imaging data to determine if one or both of this data should be used to align the reconstructed image and to what extent each should be used in the alignment process. In one embodiment, an accuracy of the positional data acquired by the sensor 108 may be determined. The imaging system 100 may determine an accuracy of the positional data. Generally, the sensor 108 has a higher level of accuracy early in the scanning process. Accordingly, if the positional data is accurate, the imaging system 100 may reconstruct the 3D image at 208 based only on the positional data. However, the positional information from the sensor 108 may be subject to drift over time. For example, over the time period of image acquisition, the positional data may become inaccurate causing blurring and/or jagged edges in the reconstructed 3D image.

The imaging module 118 may compensate for errors in the positional data using the imaging data. The imaging module 118, thus, may compensate for the errors in the positional data from the sensor 108 or tracking device 109 (e.g. correcting, adjusting, or aligning multiple 2D image slices). For example, the imaging module 118 may compensate for errors in the positional data utilizing landmarks present in the imaging data. In various embodiments, the imaging module 118 uses imaging data that is fused with the positional data by a filter, for example, a Kalman filter or other mathematical method for tracking position that forms part of the position determination module 114.

The imaging system 100 determines an accuracy of the alignment of the 3D reconstructed image, which may be determined continuously, at intervals, etc. The accuracy of the alignment of the image using the imaging data may be determined using any suitable information, for example, based also on landmarks within the image and/or image matching. For example, a comparison between images from a new 2D scan may be compared to already acquired images, for example, using the image landmarks. In one embodiment, the imaging system 100 may acquire further data by notifying the operator to continue scanning the patient for additional positional data and/or imaging data. The imaging system 100 may also determine additional positional data based on input from the sensor 108. Alternatively, the imaging system 100 may acquire additional imaging data from the imaging probe 106. In one embodiment, the imaging system 100 both determines additional positional data and acquires additional imaging data. The ratio of additional imaging data acquired to additional positional data determined may be based on the weighting ratio that is indicative of the amount of error in each of the imaging data and the positional data.

In one embodiment, the imaging system 100 automatically acquires the additional data in real time based on the quality of the reconstructed 3D image. In another embodiment, the reconstructed 3D image is displayed on the display 120 (shown in FIGS. 1 and 2) during scanning. The operator may access the reconstructed 3D image to determine additional data that may be required. If additional positional data is required, such as when filling in a reconstructed image boundary, the operator may obtain the positional data by performing broad strokes or sweeps on the patient with the imaging probe 106 and, if additional imaging data is required, the operator may obtain the imaging data by performing finer strokes or sweeps on the patient with the imaging probe 106 to focus on the region of interest.

In various embodiments, the imaging data is weighted with respect to the positional data. A weighting ratio is determined to weight errors in the positional data versus errors in the imaging data. In one embodiment, the imaging system 100 automatically varies the weighting ratio based the quality of the positional data and the imaging data, which may be based, for example, on the output of the Kalman filter. In another embodiment, more weight is given to the positional data early in the scan, and as the scan progresses, more weight is given to the imaging data. The weighting ratio may vary throughout the scan. Alternatively, the weighting ratio is automatically varied based on predetermined changes in the weighting ratio with respect to time. In another embodiment, the operator may update the weighting ratio, such as throughout the scan.

The weighting ratio may be based on noise models generated for the imaging data and the positional data. For example, as the noise in the positional data increases, the noise in the imaging data decreases. Accordingly, the weighting ratio is varied so that the imaging data is predominately used to align the reconstructed 3D image as the noise in the positional data increases.

The imaging data is then used along with the positional data to align the reconstructed 3D image at 210. For example, the aligned imaging data may be utilized to fill in a previously reconstructed 3D image boundary to complete the reconstruction of the 3D image, for example, when going from broad scanning strokes to more focused scanning strokes. The imaging data may also provide an increased level of granularity for positional information used in the image reconstruction. In various embodiments, the imaging data is used to align or correct the positional data from the sensor 108 or tracking device 109 so that the imaging probe 106 does not require recalibration during scanning.

Thus the reconstructed 3D image is aligned using a combination of the imaging data and the positional data, which may include determining which of the imaging data and positional data is more accurate with respect to positional information as determined by the data (positional data or imaging data) with the lowest error. The 3D image is, thus, reconstructed based on the true or more accurate location based on the lowest error. In one embodiment, the 3D image is reconstructed during the scan, which allows the operator interaction and input. Alternatively, the imaging system 100 collects the positional data and imaging data during the scan and processes the data after the scan. In such an embodiment, the 3D image is reconstructed post-scan by the imaging module 118. At 212, the reconstructed 3D image is displayed on display 120.

FIG. 5 is a graph 300 of root mean square data of image slices acquired by the imaging probe 106 (shown in FIGS. 1 and 2) and which may be used to correct for errors in the positional data from the sensor 108 or tracking device 109. The data shows the root mean square from a center image slice to surrounding slices. The x-axis 302 is a distance of a 2D imaging slice from a center 2D image slice. The y-axis 304 is the value of the root mean square distance of the 2D imaging slice from the center 2D image slice. Curve 306 illustrates a plurality of 2D image slices. Based on the root mean square distance of the 2D image slices from the center 2D image slice, the position determination module 114 (shown in FIGS. 1 and 2) can determine a more accurate position based on using the image slice with the lowest error, such as, by using the RMS metric and weighting the positional data accordingly. This image data may be used in combination with the determination of the similarity between images from a new scan and a previous scan. Thus, a RMS metric may be provided as follows:

$$RMS ( a , b ) = \sqrt{\frac{1}{N} {\sum }_{i}^{N} {( {a}_{i} , {b}_{i} )}^{2}}$$

Thus, the correlation between the 2D image slices may be utilized to align the images for 3D image reconstruction in combination with the positional data.

It should be noted that FIG. 5 illustrates only one image metric that may be used to align 3D images formed from free-hand 2D images. The method 200 is not limited to utilizing the root mean square data. In other embodiments, the reconstructed 3D image may be aligned using at least one of a correlation between the 2D imaging data, mutual information in the 2D imaging data, a histogram comparison of the 2D imaging data, speckle correlation, or the like.

FIG. 6 is an exemplary representation 400 of error over time from different data used in 3D image reconstruction. The x-axis 402 represents time and the y-axis 404 represents a degree of error. Curve 406 represents the error over time for 3D reconstruction using positional data determined by the sensor 108 (shown in FIGS. 1 and 2). As illustrated, the degree of error increases over time using positional data. The valleys 408 represent a time period in the scan when the sensor 108 is recalibrated by holding the imaging probe 106 (shown in FIGS. 1 and 2) still. Once the scan continues, the error in the positional data increases until the sensor 108 is recalibrated. The curve 410 represents a degree of error in imaging data acquired by the imaging probe 106 over time. As illustrated, the error in the imaging data decreases over time.

Curve 412 represents error over time in 3D image reconstruction using both the positional data and the imaging data to align and reconstruct 3D images in accordance with various embodiments. The curve 412 represents the degree of error when the imaging data is fused with the positional data as described in method 200. As illustrated, the degree of error is minimized and relatively constant when utilizing both the positional data and the imaging data.

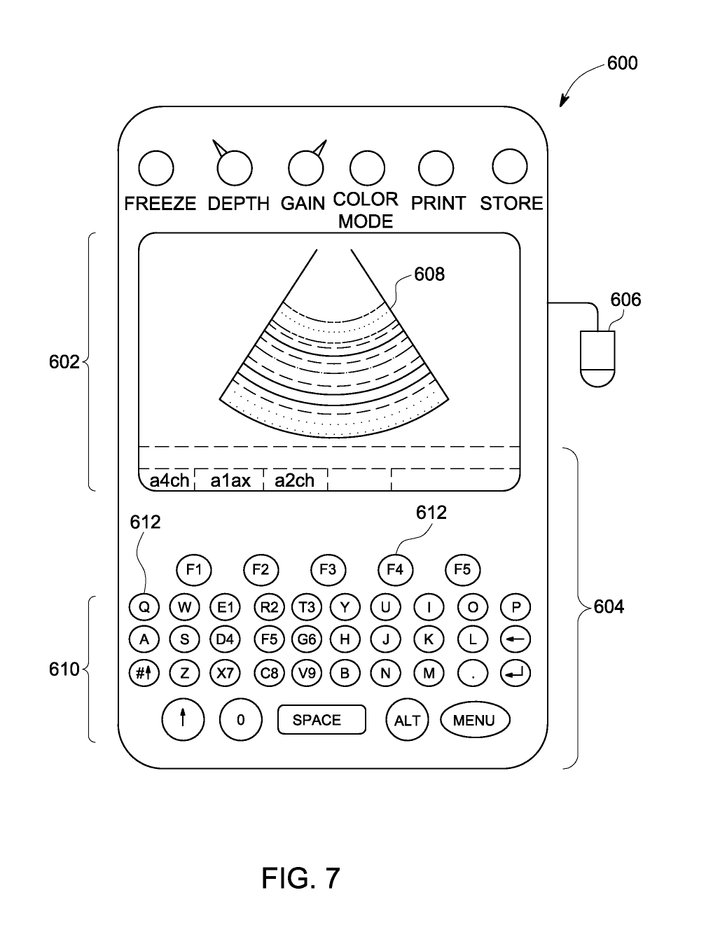

FIG. 7 illustrates a hand carried or pocket-sized ultrasound imaging system 600 (which may be embodied as the imaging system 100). The ultrasound imaging system 600 may be configured to operate as described in the method 200 (shown in FIG. 3). The ultrasound imaging system 600 has a display 602 and a user interface 604 formed in a single unit. By way of example, the ultrasound imaging system 600 may be approximately two inches wide, approximately four inches in length, and approximately half an inch in depth. The ultrasound imaging system may weigh approximately three ounces. The ultrasound imaging system 600 generally includes the display 602 and the user interface 604, which may or may not include a keyboard-type interface and an input/output (I/O) port for connection to a scanning device, for example, an ultrasound probe 606. The display 602 may be, for example, a 320×320 pixel color LCD display on which a medical image 608 may be displayed. A typewriter-like keyboard 610 of buttons 612 may optionally be included in the user interface 604.

The probe 606 may be coupled to the system 600 with wires, cable, or the like. Alternatively, the probe 606 may be physically or mechanically disconnected from the system 600. The probe 606 may wirelessly transmit acquired ultrasound data to the system 600 through an access point device (not shown), such as an antenna disposed within the system 600.

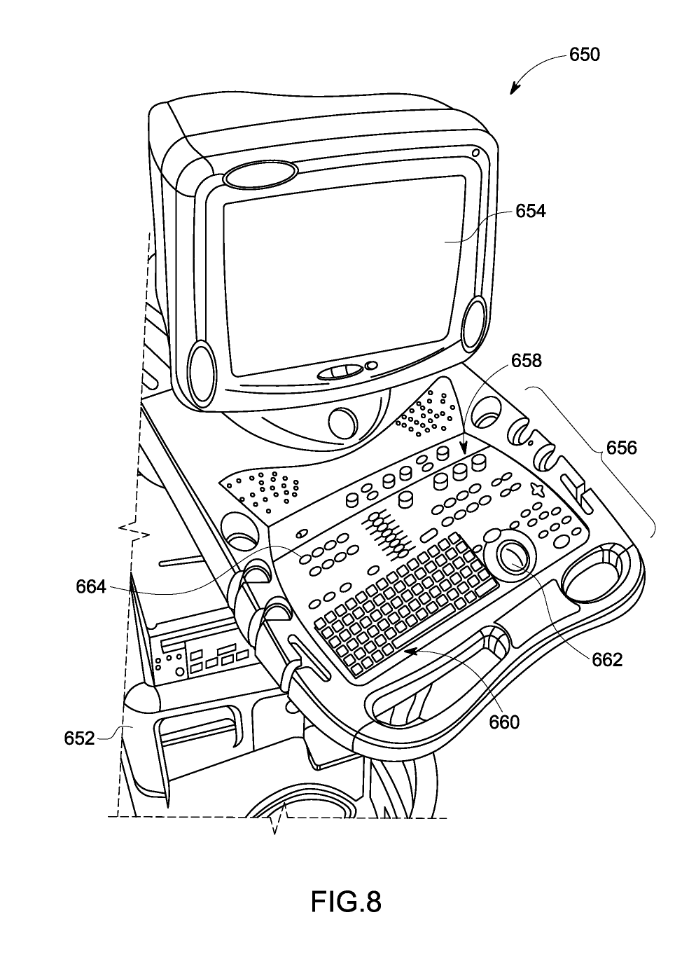

FIG. 8 illustrates an ultrasound imaging system 650 (which may be embodied as the imaging system 100) provided on a moveable base 652. The ultrasound imaging system 650 may be configured to operate as described in the method 200 (shown in FIG. 3). A display 654 and a user interface 656 are provided and it should be understood that the display 654 may be separate or separable from the user interface 656. The user interface 656 may optionally be a touchscreen, allowing an operator to select options by touching displayed graphics, icons, and the like.

The user interface 656 also includes control buttons 658 that may be used to control the system 650 as desired or needed, and/or as typically provided. The user interface 656 provides multiple interface options that the user may physically manipulate to interact with ultrasound data and other data that may be displayed, as well as to input information and set and change scanning parameters and viewing angles, etc. For example, a keyboard 660, trackball 662, and/or other multi-function controls 664 may be provided. One or more probes (such as the probe 106 shown in FIG. 1) may be communicatively coupled with the system 650 to transmit acquired ultrasound data to the system 650.

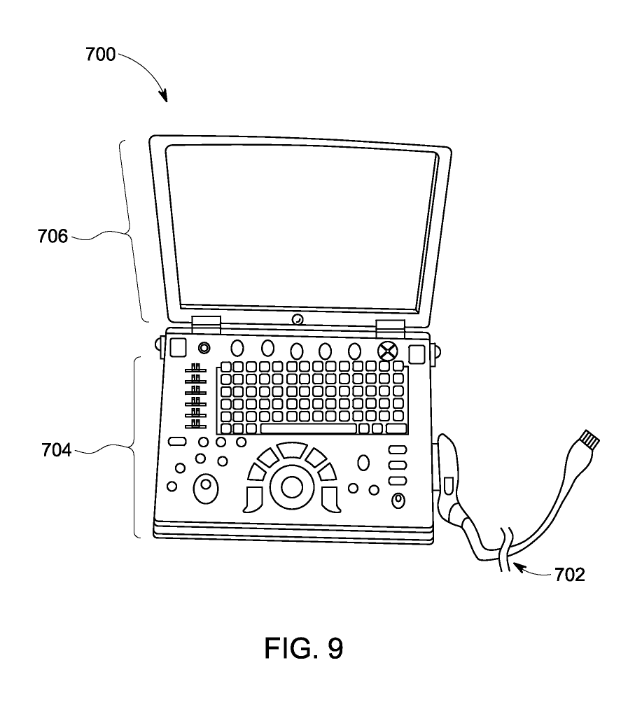

FIG. 9 illustrates a 3D-capable miniaturized ultrasound system 700 (which may be embodied as the imaging system 100). The ultrasound imaging system 700 may be configured to operate as described in the method 200 (shown in FIG. 3). The ultrasound imaging system 700 has a probe 702 that may be configured to acquire 3D ultrasonic data or multi-plane ultrasonic data. A user interface 704 including an integrated display 706 is provided to receive commands from an operator. As used herein, “miniaturized” means that the ultrasound system 700 is a handheld or hand-carried device or is configured to be carried in a person's hand, pocket, briefcase-sized case, or backpack. For example, the ultrasound system 700 may be a hand-carried device having a size of a typical laptop computer. The ultrasound system 700 is easily portable by the operator. The integrated display 706 (e.g., an internal display) is configured to display, for example, one or more medical images.

The various embodiments enable accurate reconstruction of 3D volumes from free-hand 2D ultrasound scans with low-cost position sensor. By fusing image data with positional data, a 3D image is reconstructed with continuous acquisition with no need to recalibrate the positional sensor. The combined image data and positional data enables 3D image reconstruction with less error in comparison to 3D image reconstruction utilizing only positional or image data. When the image data is utilized with the positional data, the image data can be used to refine a location of the imaging probe calculated by the sensor. In one embodiment, the image data is used to calculate a similarity of the 2D image data with already acquired image data. A true position is then indicated by the lowest error, such as by using an RMS metric.

The technical advantages of the various embodiments include combining the advantages of positional data and image data so that if one of the positional data or the image data is weak and the other is strong, a weighted combination provides more accurate image reconstructions. The various embodiments enable low-cost sensors to be used with the imaging probe. In one embodiment, there is no need for calibration of the sensors. Accordingly, the operator can continue to sweep the probe across the object of interest as long as necessary to fill the 3D volume.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, flash drive, jump drive, USB drive and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the described subject matter without departing from their scope. While the dimensions and types of materials described herein are intended to define the parameters of the various embodiments of the invention, the embodiments are by no means limiting and are exemplary embodiments. Many other embodiments will be apparent to one of ordinary skill in the art upon reviewing the above description. The scope of the various embodiments of the inventive subject matter should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments of the invention, including the best mode, and also to enable one of ordinary skill in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. An imaging system for generating three-dimensional (3D) images comprising:
an imaging probe for acquiring two-dimensional (2D) image data of a region of interest;
a sensor coupled with the imaging probe to determine positional data related to a position of the imaging probe;
a position determination module utilizing the image data acquired with the imaging probe and the positional data determined by the sensor to calculate a probe location with respect to the acquired 2D image data; and
an imaging module configured to reconstruct a 3D image of the region of interest based on the 2D image data and the determined probe locations.

2. The imaging system of claim 1, wherein the positional data determined by the sensor is used during a first scan to align reconstructed 3D image boundaries and the image data acquired by the imaging probe is used during subsequent scans to align an additional reconstructed 3D image within the reconstructed 3D image boundaries.

3. The imaging system of claim 1, wherein the positional data determined by the sensor is used during a first scan to align reconstructed 3D image boundaries and the image data acquired by the imaging probe is used during subsequent scans to increase a level of alignment granularity in the reconstructed 3D image.

4. The imaging system of claim 1, wherein the image data compensates for errors in the positional data.

5. The imaging system of claim 1, wherein the imaging module at least one of corrects, adjusts, or aligns multiple image frames based on indentified landmarks in the image data.

6. The imaging system of claim 1, wherein the imaging probe is at least one of an ultrasound probe or an infrared optical tomography probe.

7. The imaging system of claim 1, wherein the sensor includes at least one of a position tracking device, an accelerometer, or a gyroscope.

8. The imaging system of claim 1, wherein a weighting ratio of image data to positional data utilized to reconstruct the 3D image varies with respect to an amount of error within at least one of the image data or the positional data.

9. The imaging system of claim 1, wherein a weighting ratio of image data to positional data utilized to reconstruct the 3D image increases over a time period of acquiring the image data.

10. The imaging system of claim 1, wherein the 3D image is reconstructed by weighting noise from the positional data with respect to noise from the image data.

11. A method for generating three-dimensional (3D) images comprising:
acquiring two-dimensional (2D) image data of a region of interest with an imaging probe;
determining positional data related to a position of the imaging probe with a sensor coupled with the imaging probe;
calculating a probe location with respect to the acquired 2D image data with the image data acquired with the imaging probe and the positional data determined by the sensor; and
reconstructing a 3D image of the region of interest based on the 2D image data and the determined probe locations.

12. The method of claim 11 further comprising:
aligning reconstructed 3D image boundaries using the positional data determined by the sensor during a first scan; and
aligning an additional reconstructed 3D image within the reconstructed 3D image boundaries using image data acquired by the imaging probe during subsequent scans.

13. The method of claim 11 further comprising:
aligning reconstructed 3D image boundaries using the positional data determined by the sensor during a first scan; and
increasing a level of alignment granularity in the reconstructed 3D image using image data acquired by the imaging probe during subsequent scans.

14. The method of claim 11 further comprising compensating for errors in the positional data with the image data.

15. The method of claim 11 further comprising varying a weighting ratio of image data to positional data to reconstruct the 3D image.

16. The method of claim 11 further comprising increasing a weighting ratio of image data to positional data over time to reconstruct the 3D image.

17. A non-transitory computer readable storage medium for generating three-dimensional (3D) images using a processor, the non-transitory computer readable storage medium including instructions to command the processor to:
acquire two-dimensional (2D) image data of a region of interest with an imaging probe;
determine positional data related to a position of the imaging probe with a sensor coupled with the imaging probe;
calculate a probe location with respect to the acquired 2D image data with the image data acquired with the imaging probe and the positional data determined by the sensor; and
reconstruct a 3D image of the region of interest based on the 2D image data and the determined probe locations.

18. The non-transitory computer readable storage medium of claim 17, wherein the instructions command the processor to:
align reconstructed 3D image boundaries using the positional data determined by the sensor during a first scan; and
align an additional reconstructed 3D image within the reconstructed 3D image boundaries using image data acquired by the imaging probe during subsequent scans.

19. The non-transitory computer readable storage medium of claim 17, wherein the instructions command the processor to compensate for errors in the positional data with the image data.

20. The non-transitory computer readable storage medium of claim 17, wherein the instructions command the processor to vary a weighting ratio of image data to positional data to reconstruct the 3D image.
